(19) United States
(12) Patent Application Publication
(10) Pub. No.: US 2025/0381051 A1
(43) Pub. Date: Dec. 18, 2025

(54) MACHINE HUMAN INTERFACE FOR PROSTHETIC CONTROL

(71) Applicant: Wei-Chiang Lin, Miami, FL (US)

(72) Inventor: Wei-Chiang Lin, Miami, FL (US)

(73) Assignee: The Florida International University Board of Trustees, Miami, FL (US)

(21) Appl. No.: 19/235,158

(22) Filed: Jun. 11, 2025

Related U.S. Application Data
(60) Provisional application No. 63/660,024, filed on Jun. 14, 2024.

(51) Int. Cl. A61F 4/00; A61F 2/54; A61F 2/68; A61F 2/70; A61F 2/76; G06F 3/01; G06V 20/20

(52) U.S. Cl. CPC A61F 4/00; A61F 2/54; G06V 20/20; A61F 2002/6809; A61F 2002/6881; A61F 2002/704; A61F 2002/769; G06F 3/012

(57) ABSTRACT

Machine-human interface (MHI) systems for control of powered external movement devices, such as prosthetics (e.g., prosthetic hands and/or arms), are provided, as well as methods of using the same. The efficient MHI systems leverage features of computer vision and pattern recognition to examine the subjects and/or objects within the field of view of a user of the system, and then uses artificial intelligence and/or machine learning to guess the user's intention. Once the user acknowledges the guessed intention, the MHI system can measure the location of the targeted subject/object using a measuring means (e.g., using Light Detection and Ranging (LIDAR) technology) and then coordinate the movement of the external movement device (e.g., prosthetic arm and/or hand).

### CROSS-REFERENCE TO RELATED APPLICATION

This application claims the benefit of U.S. Provisional Application Ser. No. 63/660,024, filed Jun. 14, 2024, the disclosure of which is hereby incorporated by reference in its entirety, including all figures, tables, and drawings.

### BACKGROUND

Machine-human interface (MHI) systems typically utilize implantable electrodes to record neural signals and attempt to decipher a user's intention. Existing MHI systems also suffer from complexity (requiring extensive training for the user), difficulty in operation, and inaccuracies in executing movements of a prosthetic.

### BRIEF SUMMARY

Embodiments of the subject invention provide novel and advantageous machine-human interface (MHI) systems for control of powered external movement devices, such as prosthetics (e.g., prosthetic hands and/or arms). The efficient MHI systems leverage features of computer vision and pattern recognition to examine the subjects and/or objects within the field of view of a user of the system, and then uses artificial intelligence (AI) and/or machine learning to guess the user's intention. Once the user acknowledges the guessed intention, the MHI system can measure the location of the targeted subject/object using a measuring means (e.g., using Light Detection and Ranging (LIDAR) technology) and then coordinate the movement of the external movement device (e.g., prosthetic arm and/or hand).

In an embodiment, an MHI system can comprise: a wearable display; an external movement device (e.g., a prosthetic, a robotic arm for an able-bodied user, or a machine for an able-bodied user); a microcontroller unit (MCU) in operable communication with both the wearable display and the external movement device; and a machine-readable medium in operable communication with the MCU and having instructions stored thereon that, when executed by the MCU, perform the following steps: i) acquiring an image of a field of view of a user of the system using the wearable display; ii) identifying at least one object within the field of view of the user; iii) predicting an intention of the user based on the at least one object within the field of view of the user; iv) providing to the user, via the wearable display, a list of actions relevant to the at least one object within the field of view of the user; v) receiving input from the user regarding the list of actions; and vi) sending a command to control the external movement device based on the input from the user regarding the list of actions. The wearable display can comprise a camera module and a heads-up display. The heads-up display can be configured to provide an augmented reality (AR) function. The camera module can comprise: a wide-angle image sensor configured to capture at least a majority of a view of the user; and/or a LIDAR camera configured to generate an accurate three-dimensional (3D) view of the field of view of the user. The predicting of the intention of the user can comprise using AI and/or machine learning. The AI (and/or machine learning) can have self-learning capabilities, such that it can learn from incorrect predictions and improve future predictions, thereby becoming more personalized to a given user over time. The MHI system can further comprise a microphone and/or a motion sensor in operable communication with the MCU, and the receiving of the input from the user can comprise receiving voice input from the user via the microphone and/or receiving input from the motion sensor (e.g., receiving head movement input, such as head nodding to indicate an affirmative response or shaking the head to indicate a negative response, via the motion sensor). The command to control the external movement device can comprise a command for the external movement device to interact with the at least one object. The wearable display can comprise, for example, glasses. The external movement device can comprise, for example, a prosthetic hand, a prosthetic arm, and/or a robotic arm. The MHI system can exclude any implantable components (i.e., the MHI system can include no components that are implantable or implanted in the user), such that the MHI system is completely non-invasive to the user.

In another embodiment, a method for controlling an external movement device (e.g., a prosthetic, a robotic arm for an able-bodied user, or a machine for an able-bodied user) can comprise: i) providing an MHI system as described herein (including any or all of the features described in the previous paragraph); ii) acquiring an image of a field of view of a user of the system using the wearable display; iii) identifying at least one object within the field of view of the user; iv) predicting an intention of the user based on the at least one object within the field of view of the user; v) providing to the user, via the wearable display, a list of actions relevant to the at least one object within the field of view of the user; vi) receiving input from the user regarding the list of actions; and vii) controlling the external movement device based on the input from the user regarding the list of actions. The heads-up display can provide an AR function. The camera module can comprise: a wide-angle image sensor capturing at least a majority a view of the user; and a LIDAR camera generating an accurate 3D view of the field of view of the user. The predicting of the intention of the user can comprise using AI and/or machine learning. The AI (and/or machine learning) can have self-learning capabilities, such that it can learn from incorrect predictions and improve future predictions, thereby becoming more personalized to a given user over time. The receiving of the input from the user can comprise receiving voice input from the user via a microphone of the MHI system and/or receiving input from a motion sensor of the MHI system (e.g., receiving head movement input, such as head nodding to indicate an affirmative response or shaking the head to indicate a negative response, via the motion sensor). The command to control the external movement device can comprise a command for the external movement device to interact with the at least one object.

### BRIEF DESCRIPTION OF DRAWINGS

FIG. 1 shows a schematic view of a machine-human interface (MHI) system, according to an embodiment of the subject invention.

FIG. 2 shows a schematic view of an MHI system, according to an embodiment of the subject invention.

FIG. 3 shows a functional block diagram of an MHI system, according to an embodiment of the subject invention.

FIG. 4 shows a functional block diagram of an MHI system, according to an embodiment of the subject invention.

FIG. 5 shows a functional block diagram of an MHI system, according to an embodiment of the subject invention.

FIG. 6 shows a functional block diagram of an MHI system, according to an embodiment of the subject invention.

FIG. 7 shows a functional block diagram of an MHI system, according to an embodiment of the subject invention.

### DETAILED DESCRIPTION

Embodiments of the subject invention provide novel and advantageous machine-human interface (MHI) systems for control of powered external movement devices, such as prosthetics (e.g., prosthetic hands and/or arms). The MHI system can also be used by a user with no prosthetic, such as by an able-bodied user controlling, e.g., a robotic arm and/or a machine. The efficient MHI systems leverage features of computer vision and pattern recognition to examine the subjects and/or objects within the field of view of a user of the system, and then uses artificial intelligence (AI) and/or machine learning to guess the user's intention. Once the user acknowledges the guessed intention, the MHI system (which can be referred to herein as simply “the MHI”) can measure the location of the targeted subject/object using a measuring means (e.g., using Light Detection and Ranging (LIDAR) technology) and then coordinate the movement of the external movement device (e.g., prosthetic arm and/or hand).

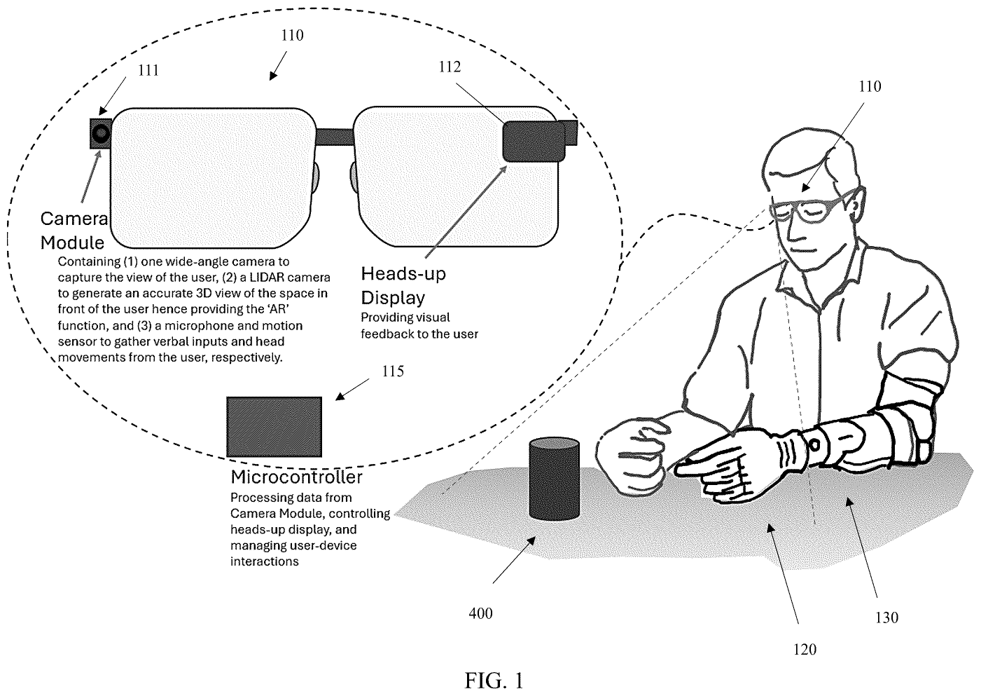

FIG. 1 shows a schematic view of an MHI system, according to an embodiment of the subject invention. Referring to FIG. 1, the system can include a wearable display 110 (e.g., a pair of glasses), an external movement device 120,130 (e.g., a prosthetic hand 120 and/or arm 130), and a microcontroller unit (MCU) 115 in operable communication with both the glasses 110 and the external movement device 120,130. For convenience, the wearable display 110 can be referred to herein as the pair of glasses or the glasses (but the wearable display is not necessarily limited to glasses). The MCU can be disposed on (or in) the glasses 110, on (or in) the external movement device 120,130, or elsewhere (so long as it is in operable communication with the glasses 110 and the external movement device 120,130). The glasses 110 can be augmented reality (AR) glasses and can include a camera module 111 (e.g., a 4k camera module) and a heads-up display 112 configured to provide visual feedback to the user. The camera module 111 can include: a wide-angle camera configured to capture most or all of the view of the user; a LIDAR camera configured to generate an accurate three-dimensional view of the space in front of the user (i.e., providing an AR function); a microphone configured to receive verbal input from the user; and/or a motion sensor configured to receive head motion input (e.g., head nodding to indicate an affirmative response by the user or shaking of the head to indicate a negative response by the user). Any of these components of the camera module 111 can be located on other portions of the glasses 110 or even other portions of the system. For example, the microphone and/or the motion sensor can be disposed on the external movement device 120,130, so long as it is, or they are, in operable communication with the MCU 115. The MCU 115 is configured to process data from the camera module 111, control the heads-up display 112, and mange user-device interactions. The MCU 115 can also control the external movement device 120,130 and/or control the AI that is used to guess the user's intention. The AI (and/or machine learning) can have self-learning capabilities, such that it can learn from incorrect predictions and improve future predictions, thereby becoming more personalized to a given user over time. The system can include a (non-transitory) computer-readable medium (not pictured) in operable communication with the MCU 115 and having software (e.g., one or more modules) stored thereon that performs some or all of the control functions (e.g., AI, controlling the external movement device 120,130, controlling the heads-up display 112, analyzing the camera module 111 input, etc.).

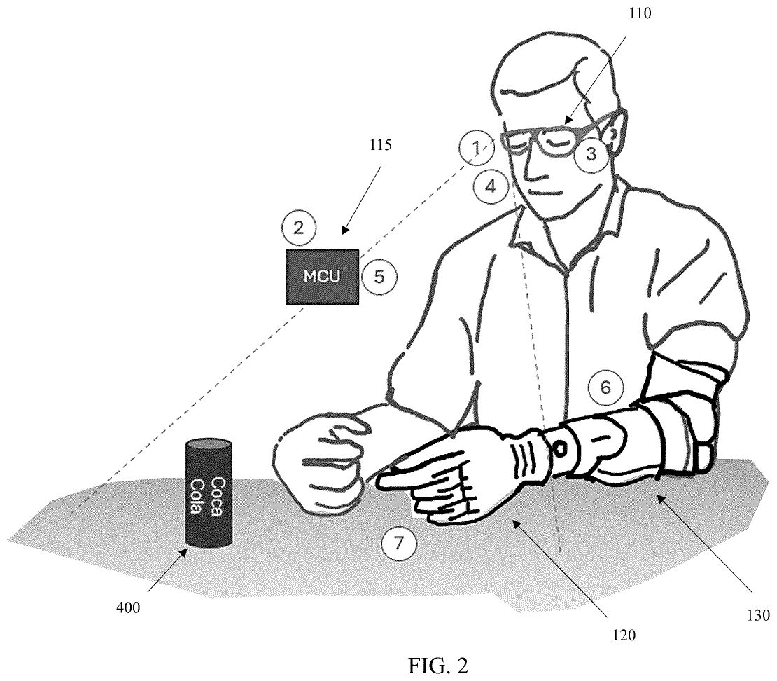

FIG. 2 shows a schematic view of an MHI system, according to an embodiment of the subject invention, including circled numbers for identification of certain aspects. The circled numbers (1 through 7) in FIG. 2 correspond to the labeled aspects in this paragraph. (1) The wide-angle camera in the camera module 111 can capture most or all of the field of view of the user, and the LIDAR camera in the camera module 111 can produce a 3D view of the same field. (2) The software can process the images captured by the wide-angle camera and identify unique objects or subjects (e.g., a can 400) within the view using AI. (3) The software can posit a question about the object 400 (e.g., do you want to drink the contents of the can?) on the heads-up display 112. (4) The computer software can get the response from the user via the microphone in the camera module 111 and/or via the motion sensor in the camera module 111. (5) If the user's response to the question is affirmative (i.e., the user confirms that the AI prediction was correct), the software processes the 3D data from the LIDAR camera and determines the coordinates of the targeted object 400. (6) The software can send the action command/object coordinates to a controller (which may be the MCU 115 or may be a different controller) of the external movement device 120,130. (7) The controller of the external movement device 120,130 can execute the action via the assistance of the LIDAR camera information (e.g., the coordinates of the object 400 and the prosthetic hand 120).

Embodiments of the subject invention include methods of using the MHI systems disclosed herein. A method can include providing the MHI system and using it according to its intended purpose to assist the user in operating the external movement device via AI.

The systems of embodiments of the subject invention may have no invasive components (e.g., electrodes), which are typical in related art and experimental MHIs. The systems of embodiments of the subject invention are also faster, more reliable, and more accurate than related MHIs. The incorporation of the measuring means (e.g., LIDAR technology) can significantly improve the accuracy and user experience of using powered external movement devices (e.g., prosthetic hands and arms). The MHI system can include a wearable display with a wide-field camera and a LIDAR camera embedded therein.

The user can wear the glasses with a camera module including a wide-field camera and a LIDAR camera embedded therein. The wide-field camera can record the user's view continuously, and software can analyze the images from the camera module to identify unique subjects or objects within the view. From the identified subjects, the software can make an intelligent prediction of the user's intention and then show it on the heads-up display of the display. The user can provide verbal (to be received by the microphone) or motion (to be received by one or more of the cameras and/or the motion sensor) feedback (e.g., yes or no) to the prediction. If the user positively confirms the prediction, the LIDAR camera can measure the spatial location of the targeted subject/object with respect to the user, and then coordinate the movement of the external movement device to complete the action toward the targeted subject/object. If the user indicates that the prediction is not correct, the system will provide an updated (different) intelligent prediction of the user's intention and the process is repeated (user provides feedback, system responds to the feedback from the user).

One important advantage of the MHI systems of embodiments of the subject invention is that they are non-invasive. That is, they do not rely on implantable devices (e.g., implantable electrodes) to record neural signals to decipher the user's intention. With computer technologies such as image/pattern recognition, intention prediction, and 3D measurements, the MHI systems of embodiments of the subject invention can deliver higher accuracy and shorter response time than related art MHIs such as electroencephalogram (EEG)-based and electromyography (EMG)-based controllers. In addition, the LIDAR camera can provide real-time feedback on the operation of the external movement device, which increases the accuracy and precision of the action(s) of the external movement device. The MHI systems are easy to use, such that the users thereof would not be expected to face a steep learning curve.

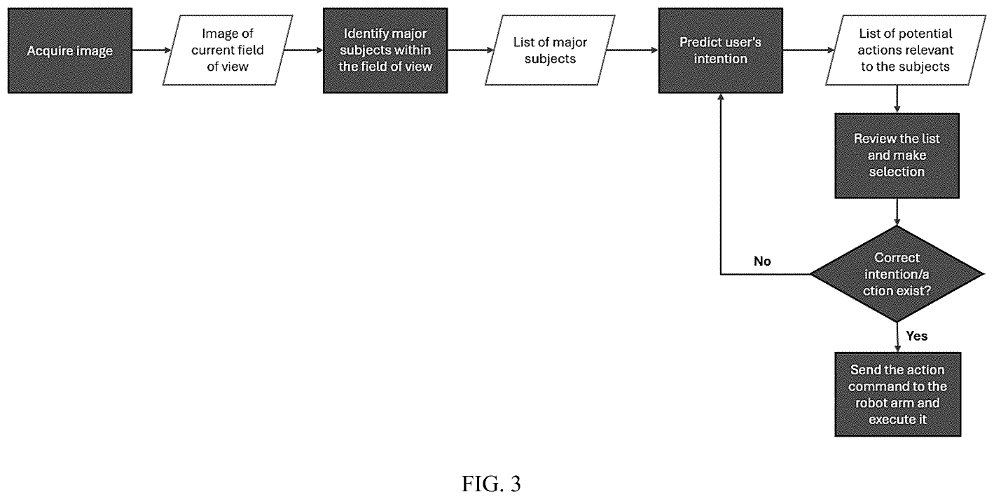

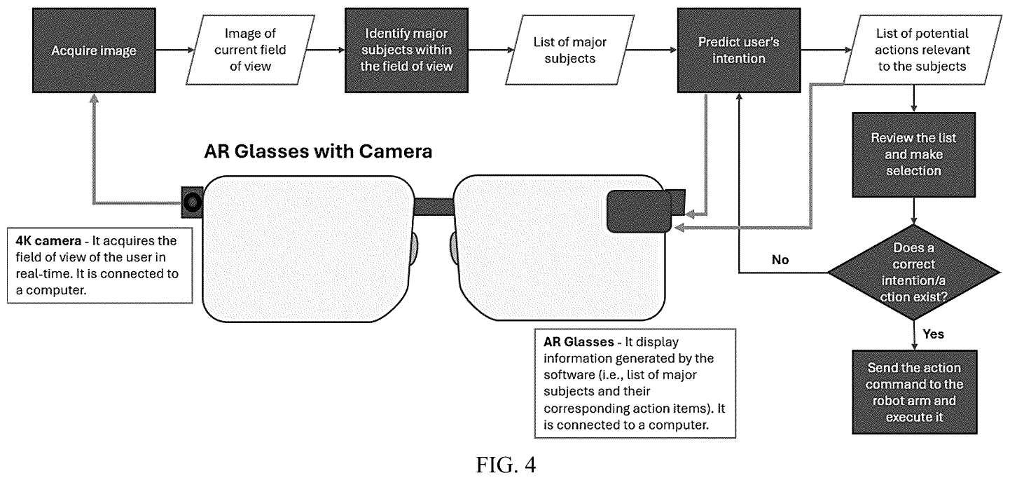

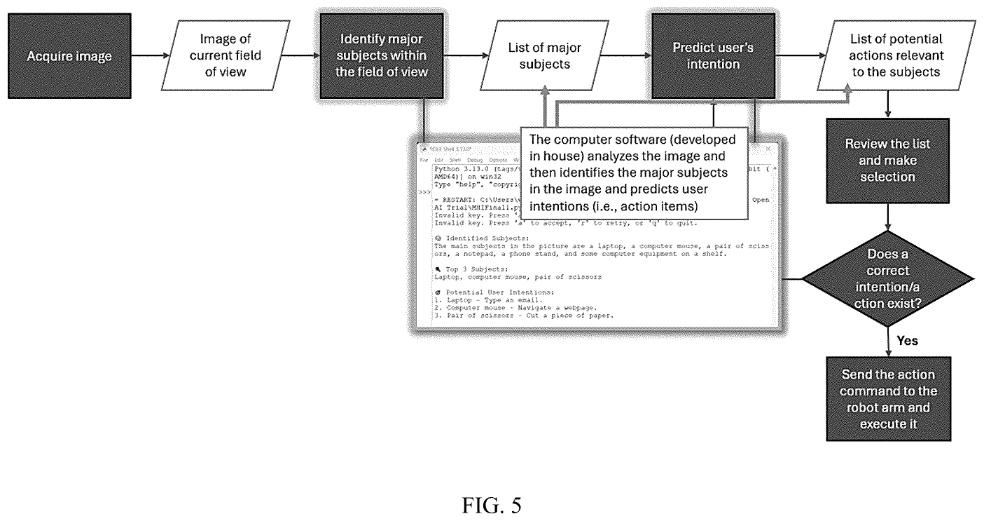

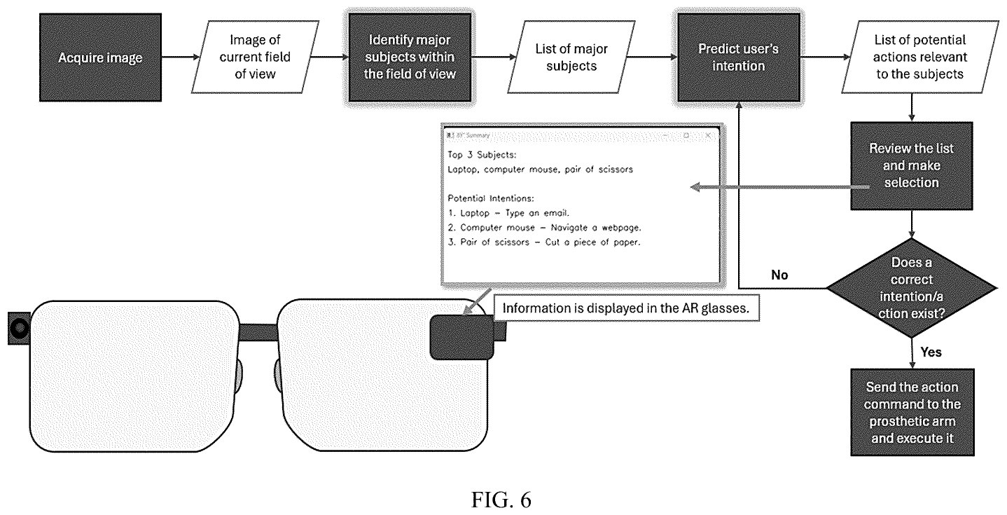

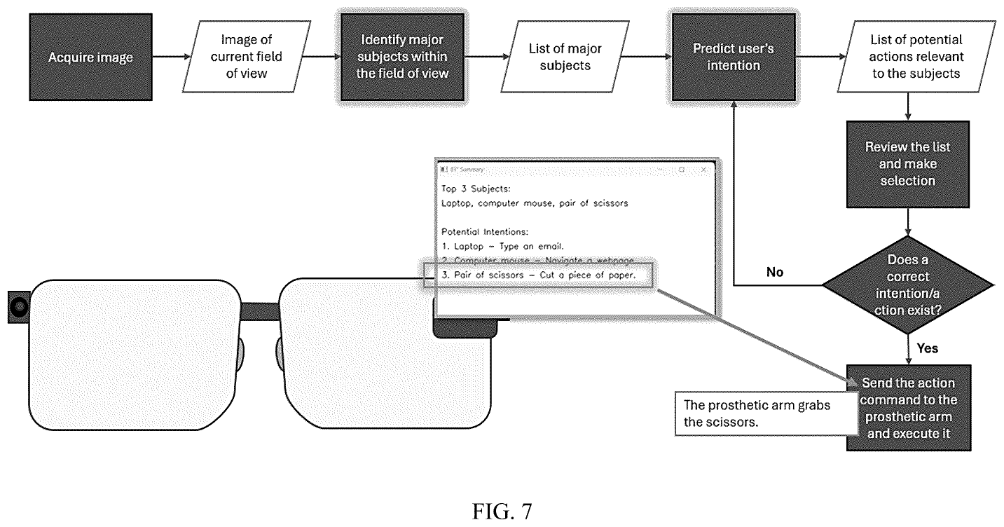

FIGS. 3-7 show functional block diagrams of embodiments of the subject invention. Referring to these figures, an MHI can acquire (e.g., using the camera module 111) an image, identify major subjects/objects, predict (e.g., by the MCU 115) the user's intention, provide (e.g., via the heads-up display 112) a list of possible intentions to the user, receive (e.g., via the microphone and/or the motion sensor) input from the user (e.g., a selection from the list or an indication that no items on the list are a correct prediction of intention), and control the external movement device based on the input/selection of the user. If none of the items on the list correctly predict the user's intention, the MCU 115 can predict the user's intention again with this information from the user and then repeat the remainder of the process.

Embodiments of the subject invention provide a focused technical solution to the focused technical problem of how to non-invasively operate an external movement device utilizing an MHI. The solution is provided by leveraging features of computer vision and pattern recognition and using Al and/or machine learning to predict the user's intention. Once the user acknowledges a predicted intention, the MHI system can measure the location of the targeted subject/object and coordinate the movement of the external movement device. This technical solution is specific to MHI technology, addresses a technical problem within the field of MHI technology, and results in improved MHIs by avoiding the need for invasive components. Embodiments of the subject invention have the focused, technologically-specific practical application of operating a external movement device non-invasively using an MHI. In addition, the tangible elements of the wearable display 110 (including the camera module 111 and the heads-up display 112) and the external movement device 120,130 are crucial components to embodiments of the subject invention without which the system could not function.

The methods and processes described herein can be embodied as code and/or data. The software code and data described herein can be stored on one or more machine-readable media (e.g., computer-readable media), which may include any device or medium that can store code and/or data for use by a computer system. When a computer system and/or processor reads and executes the code and/or data stored on a computer-readable medium, the computer system and/or processor performs the methods and processes embodied as data structures and code stored within the computer-readable storage medium.

It should be appreciated by those skilled in the art that computer-readable media include removable and non-removable structures/devices that can be used for storage of information, such as computer-readable instructions, data structures, program modules, and other data used by a computing system/environment. A computer-readable medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); network devices; or other media now known or later developed that are capable of storing computer-readable information/data. Computer-readable media should not be construed or interpreted to include any propagating signals. A computer-readable medium of embodiments of the subject invention can be, for example, a compact disc (CD), digital video disc (DVD), flash memory device, volatile memory, or a hard disk drive (HDD), such as an external HDD or the HDD of a computing device, though embodiments are not limited thereto. A computing device can be, for example, a laptop computer, desktop computer, server, cell phone, or tablet, though embodiments are not limited thereto.

When the term module is used herein, it can refer to software and/or one or more algorithms to perform the function of the module; alternatively, the term module can refer to a physical device configured to perform the function of the module (e.g., by having software and/or one or more algorithms stored thereon).

When ranges are used herein, combinations and subcombinations of ranges (including any value or subrange contained therein) are intended to be explicitly included. When the term “about” or “approximately” is used herein, in conjunction with a numerical value, it is understood that the value can be in a range of 95% of the value to 105% of the value, i.e. the value can be +/−5% of the stated value. For example, “about 1 kg” means from 0.95 kg to 1.05 kg.

It should be understood that the examples and embodiments described herein are for illustrative purposes only and that various modifications or changes in light thereof will be suggested to persons skilled in the art and are to be included within the spirit and purview of this application.

All patents, patent applications, provisional applications, and publications referred to or cited herein are incorporated by reference in their entirety, including all figures and tables, to the extent they are not inconsistent with the explicit teachings of this specification.

What is claimed is:

1. A machine-human interface (MHI) system, comprising:
a wearable display;
an external movement device;
a microcontroller unit (MCU) in operable communication with both the wearable display and the external movement device; and
a machine-readable medium in operable communication with the MCU and having instructions stored thereon that, when executed by the MCU, perform the following steps:
i) acquiring an image of a field of view of a user of the system using the wearable display;
ii) identifying at least one object within the field of view of the user;
iii) predicting an intention of the user based on the at least one object within the field of view of the user;
iv) providing to the user, via the wearable display, a list of actions relevant to the at least one object within the field of view of the user;
v) receiving input from the user regarding the list of actions; and
vi) sending a command to control the external movement device based on the input from the user regarding the list of actions.

2. The MHI system according to claim 1, the wearable display comprising a camera module and a heads-up display.

3. The MHI system according to claim 2, the heads-up display being configured to provide an augmented reality (AR) function.

4. The MHI system according to claim 2, the camera module comprising:
a wide-angle image sensor configured to capture at least a majority of a view of the user; and
a Light Detection and Ranging (LIDAR) camera configured to generate an accurate three-dimensional (3D) view of the field of view of the user.

5. The MHI system according to claim 1, the predicting of the intention of the user comprising using artificial intelligence (AI).

6. The MHI system according to claim 1, further comprising at least one of:
a microphone in operable communication with the MCU; and
a motion sensor in operable communication with the MCU,
the receiving of the input from the user comprising at least one of: receiving voice input from the user via the microphone; and receiving head movement input from the user via the motion sensor.

7. The MHI system according to claim 1, the command to control the external movement device comprising a command for the external movement device to interact with the at least one object.

8. The MHI system according to claim 1, the wearable display comprising glasses.

9. The MHI system according to claim 1, the external movement device comprising at least one of a prosthetic hand and a prosthetic arm.

10. The MHI system according to claim 1, the MHI system excluding any implantable components, such that the MHI system is completely non-invasive to the user.

11. A method for controlling an external movement device, the method comprising:
i) providing a machine-human interface (MHI) system comprising a wearable display and the external movement device;
ii) acquiring an image of a field of view of a user of the system using the wearable display;
iii) identifying at least one object within the field of view of the user;
iv) predicting an intention of the user based on the at least one object within the field of view of the user;
v) providing to the user, via the wearable display, a list of actions relevant to the at least one object within the field of view of the user;
vi) receiving input from the user regarding the list of actions; and
vii) controlling the external movement device based on the input from the user regarding the list of actions.

12. The method according to claim 11, the wearable display comprising a camera module and a heads-up display.

13. The method according to claim 12, the heads-up display providing an augmented reality (AR) function.

14. The method according to claim 12, the camera module comprising:
a wide-angle image sensor capturing at least a majority a view of the user; and
a Light Detection and Ranging (LIDAR) camera generating an accurate three-dimensional (3D) view of the field of view of the user.

15. The method according to claim 11, the predicting of the intention of the user comprising using artificial intelligence (AI).

16. The method according to claim 11, the receiving of the input from the user comprising at least one of: receiving voice input from the user via a microphone of the MHI system; and receiving head movement input from the user via a motion sensor of the HMI system.

17. The method according to claim 11, the command to control the external movement device comprising a command for the external movement device to interact with the at least one object.

18. The method according to claim 11, the wearable display comprising glasses, and
the external movement device comprising at least one of a prosthetic hand and a prosthetic arm.

19. The method according to claim 11, the MHI system excluding any implantable components, such that the MHI system is completely non-invasive to the user.

20. A machine-human interface (MHI) system, comprising:
a wearable display;
a prosthetic;
a microphone;
a motion sensor;
a microcontroller unit (MCU) in operable communication with the wearable display, the prosthetic, the microphone, and the motion sensor; and
a machine-readable medium in operable communication with the MCU and having instructions stored thereon that, when executed by the MCU, perform the following steps:
i) acquiring an image of a field of view of a user of the system using the wearable display;
ii) identifying at least one object within the field of view of the user;
iii) predicting an intention of the user based on the at least one object within the field of view of the user;
iv) providing to the user, via the wearable display, a list of actions relevant to the at least one object within the field of view of the user;
v) receiving input from the user regarding the list of actions; and
vi) sending a command to control the prosthetic based on the input from the user regarding the list of actions,
the wearable display comprising a camera module and a heads-up display, the heads-up display being configured to provide an augmented reality (AR) function, the camera module comprising:
a wide-angle image sensor configured to capture at least a majority of a view of the user; and
a Light Detection and Ranging (LIDAR) camera configured to generate an accurate three-dimensional (3D) view of the field of view of the user, the predicting of the intention of the user comprising using artificial intelligence (AI),
the receiving of the input from the user comprising at least one of: receiving voice input from the user via the microphone; and receiving head movement input from the user via the motion sensor,
the command to control the prosthetic comprising a command for the prosthetic to interact with the at least one object,
the wearable display comprising glasses,
the prosthetic comprising at least one of a prosthetic hand and a prosthetic arm, and
the MHI system excluding any implantable components, such that the MHI system is completely non-invasive to the user.
